(19) United States
(12) Patent Application Publication
(10) Pub. No.: US 2016/0157729 A1
(43) Pub. Date: Jun. 9, 2016

(54) ACOUSTIC IMAGING APPARATUS

(71) Applicant: CANON KABUSHIKI KAISHA, Tokyo (JP)

(72) Inventor: Naoto Abe, Machida-shi (JP)

(21) Appl. No.: 14/958,757

(22) Filed: Dec. 3, 2015

(30) Foreign Application Priority Data
Dec. 9, 2014 (JP) 2014-249408
Aug. 27, 2015 (JP) 2015-168285

(51) Int. Cl. A61B 5/00

(52) U.S. Cl. CPC A61B 5/0095; A61B 5/4312; A61B 5/708; A61B 2019/5206; A61B 2562/028; A61B 2576/00

(57) ABSTRACT

An acoustic imaging apparatus includes an image forming unit including a transducer array that includes a downwardly recessed portion on which a plurality of transducers, which include reception surfaces that receive acoustic waves generated by a subject, are arranged. The image forming unit forms an image based on outputs from the transducers. Impedance matching liquid is disposed on the inner side of the transducer array. A cover member is connected to the vessel and covers the transducer array at a side opposite to a side at which the reception surfaces are arranged so that a vessel and the cover member form a housing space that accommodates the transducers. The transducers are provided with signal lines through which electric signals based on the received acoustic waves are output and which extend from the housing space to the outside through a waterproof member.

### BACKGROUND OF THE INVENTION

1. Field of the Invention

The present invention relates to an acoustic imaging apparatus, such as a photoacoustic imaging apparatus or a thermoacoustic scanner apparatus, which forms an image based on acoustic waves generated in a biological tissue.

2. Description of the Related Art

Photoacoustic imaging apparatuses for imaging the inside of living bodies by utilizing the photoacoustic effect have been studied and developed. A photoacoustic imaging apparatus irradiates the inside of a living body with pulsed laser light having a short emission period (laser pulses) and forms an image based on ultrasonic waves (photoacoustic waves) generated when a biological tissue absorbs the energy of the pulsed laser light and cubically expands due to generated heat (see Japanese Patent Laid-Open No. 2012-179348).

Thermoacoustic scanner apparatuses which form an image based on acoustic waves generated in response to a thermal expansion of a biological tissue when electromagnetic radiation pulses are applied have also been studied and developed (see U.S. Patent Application Publication No. 2011/306865).

The apparatuses described in Japanese Patent Laid-Open No. 2012-179348 and U.S. Patent Application Publication No. 2011/306865 both have a structure which includes a hemispherical transducer array and in which the space between a subject to be examined and the transducer array is filled with impedance matching liquid. The hemispherical transducer array is caused to move or rotate while receiving, with multiple transducers, acoustic waves generated in response to a thermal expansion of a biological tissue when laser pulses or electromagnetic radiation pulses are applied. A tomographic image of the subject is reconstructed from signals of the received acoustic waves.

In the case where water is used as the impedance matching liquid, the water may spill over from the transducer array when the transducer array moves or rotates. Accordingly, there is a risk that malfunction of the transducers, such as a change in signal intensity, will occur as a result of an increase in the humidity in the area surrounding the transducers or adhesion of the water to the transducers. There is also a risk that the reception performance of the transducers will be degraded due to corrosion or ion migration. To prevent this, the transducers need to be waterproofed, and the transducers and signal lines need to be kept dry. Also when liquid other than water, such as gel or castor oil, is used as the impedance matching liquid, there is a risk that a temperature change of the impedance matching liquid or the apparatus will cause dew condensation in a region outside the transducer array, that is, near the signal lines that transmit outputs from the transducers.

### SUMMARY OF THE INVENTION

An acoustic imaging apparatus according to a first aspect of the present invention includes an image forming unit including a vessel that accommodates impedance matching liquid and a transducer array that is disposed on a wall of the vessel and on which a plurality of transducers are arranged, the transducers including reception surfaces that receive acoustic waves generated by a subject, the image forming unit forming an image based on outputs from the transducers; and a cover member that covers the transducer array at a side opposite to a side at which the reception surfaces are arranged so that the vessel and the cover member form a housing space that accommodates the transducers, the cover member being connected to the vessel, the transducers being provided with signal lines through which electric signals based on the received acoustic waves are output. The signal lines extend from the housing space to the outside through a waterproof member.

An acoustic imaging apparatus according to another aspect of the present invention includes a container-shaped detector on which a plurality of ultrasonic transducers that receive acoustic waves generated by a subject are arranged. The acoustic imaging apparatus forms an image based on outputs from the ultrasonic transducers. An impedance matching material is disposed in the detector and a container-shaped cover member is disposed outside the detector, the detector and the cover member forming a sealed space. Signal lines of the ultrasonic transducers extend from the sealed space to the outside through a first waterproof member.

Further features of the present invention will become apparent from the following description of exemplary embodiments with reference to the attached drawings.

### BRIEF DESCRIPTION OF THE DRAWINGS

FIGS. 1A and 1B are schematic diagrams illustrating the structure of a photoacoustic imaging apparatus according to a first embodiment of the present invention.

FIGS. 2A to 2C are schematic diagrams illustrating a first waterproof member according to the first embodiment of the present invention.

FIG. 3 is a schematic diagram illustrating a second waterproof member according to the first embodiment of the present invention.

FIGS. 4A and 4B are schematic diagrams illustrating a modification of the first embodiment of the present invention.

FIGS. 5A and 5B are schematic diagrams illustrating the structure of a photoacoustic imaging apparatus according to a second embodiment of the present invention.

FIGS. 6A and 6B are schematic diagrams illustrating a modification of the second embodiment of the present invention.

FIGS. 7A and 7B are schematic diagrams illustrating another modification of the second embodiment of the present invention.

FIGS. 8A and 8B are schematic diagrams illustrating the structure of a photoacoustic imaging apparatus according to a third embodiment of the present invention.

FIGS. 9A and 9B are schematic diagrams illustrating a modification of the third embodiment of the present invention.

FIGS. 10A and 10B are schematic diagrams illustrating the structure of a photoacoustic imaging apparatus according to a fourth embodiment of the present invention.

FIGS. 11A and 11B are schematic diagrams illustrating the structure of a photoacoustic imaging apparatus according to a fifth embodiment of the present invention.

FIG. 12 is a block diagram of an electrical circuit of a photoacoustic imaging apparatus according to the present invention.

FIG. 13 is a block diagram illustrating the structure of a CMUT probe according to the present invention.

FIG. 14 is a circuit diagram of a relay board according to the present invention.

FIG. 15 is a schematic diagram illustrating the positions of an examination table and an examinee during an examination according to the present invention.

FIG. 16 is a schematic diagram illustrating the structure of a breast examination section of a photoacoustic imaging apparatus according to the present invention.

FIG. 17 is a schematic sectional view of an internal waterproof structure for a cup portion and a CMUT probe of a detector according to the present invention.

### DESCRIPTION OF THE EMBODIMENTS

Embodiments of the present invention will now be described. In the present invention, the term “waterproof” means not only prevention of adhesion of water to transducers, but also moisture proofing, which is prevention of an increase in humidity in a housing space.

### Structure of Examination Section of Photoacoustic Imaging Apparatus

A photoacoustic imaging apparatus for examining the human breast, to which the present invention may be advantageously applied, will be described as an embodiment of the present invention; however, the present invention is also applicable to apparatuses other than photoacoustic imaging apparatuses.

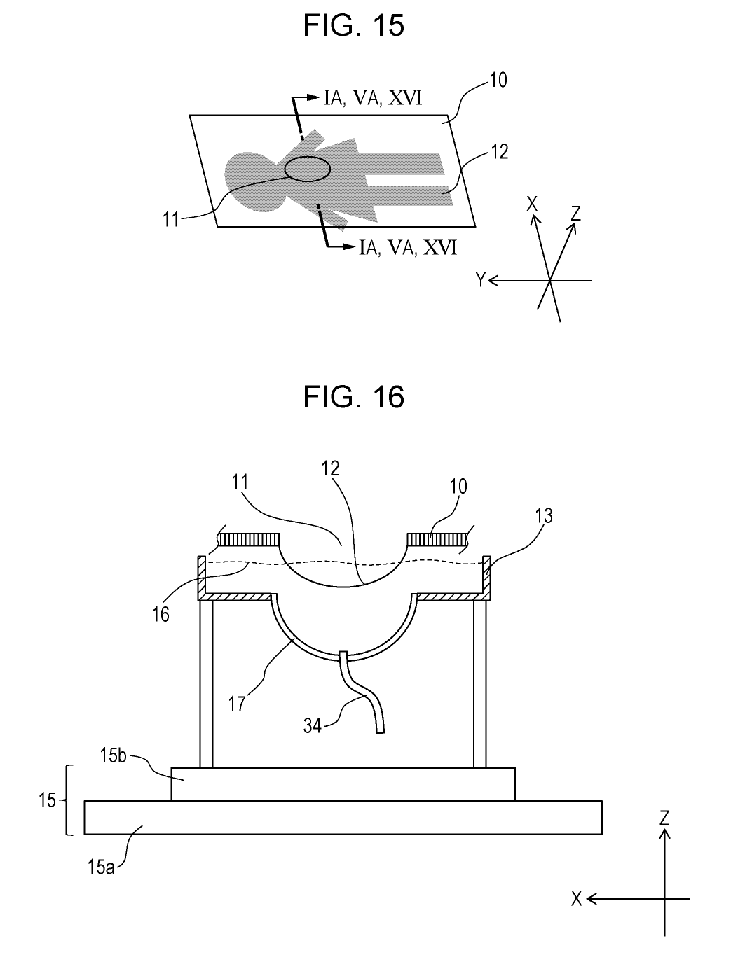

FIG. 15 is a schematic diagram illustrating the positions of an examination table and an examinee during an examination. The left-right direction, the head-toe direction, and the front-back direction of the examinee are defined as the X-axis, Y-axis, and Z-axis, respectively. A support base 10 that supports the examinee includes a vessel 13 having an insertion opening 11 into which the breast is inserted as an examination subject 1. The short-side direction of the vessel 13 may be regarded as the X-axis (horizontal plane), the longitudinal direction of the support member as the Y-axis (horizontal plane), and the thickness direction of the vessel 13 as the Z-axis.

The photoacoustic imaging apparatus according to the present embodiment examines the breast while the examinee is lying in a prone position. However, the present invention is not limited to this, and the examination may be performed while the examinee is in, for example, a sitting position. FIG. 16 illustrates the structure of a breast examination section located below the insertion opening 11, and is a schematic sectional view taken along line XVI-XVI in FIG. 15. The left-right and top-bottom directions in FIG. 16 are the X-axis and Z-axis, respectively, and the direction perpendicular to the plane of FIG. 16 is the Y-axis. A transducer array 17 includes a cup portion having a downwardly recessed portion and a plurality of transducers attached to the cup portion, and is located below the insertion opening 11. The transducers are arranged such that reception surfaces thereof are at different angles. The transducers may be arranged spherically so as to be directed toward the center of a spherical surface. When the transducer array 17 including the transducers arranged spherically around the subject is used, the reception surfaces of the transducers have large solid angles with respect to the subject. Accordingly, the sensitivity to the subject is increased, and the quality of the reconstructed image is improved.

Considering the hardware cost and the amount of calculation in reconstruction signal processing, it is not practical to increase the number of transducers without restriction. For example, when the subject 1 is the breast and the transducer array 17 is hemispherical, the radius needs to be as large as about 150 mm. In the case where the transducer array 17 includes 256 transducers, one transducer is disposed in about every 550 mm². In other words, the transducers need to be arranged at a pitch of about 23 mm.

An optical waveguide 34, such as an optical fiber, is provided. The optical waveguide 34 may instead be composed of an articulated mirror unit in which a plurality of mirrors are combined. A transducer-array scanning unit 15 includes, for example, an X stage 15a and a Y stage 15b, which each include a stepping motor and a ball screw and which are stacked on top of each other. Thus, the transducer array 17 can be freely moved in the X and Y directions. The space between the transducer array 17 and a breast holder 12 is filled with impedance matching liquid (matching layer) for achieving acoustic impedance matching. The impedance matching liquid may have a liquid surface 16 that is near the support base 10 that supports the examinee. Although not illustrated, the space between the breast holder 12 and the breast (not shown) may also be filled with the impedance matching liquid. The impedance matching liquid is liquid that has an acoustic impedance close to those of the breast and the transducers and transmits pulsed light. More specifically, water, castor oil, gel, or the like may be used.

### First Embodiment

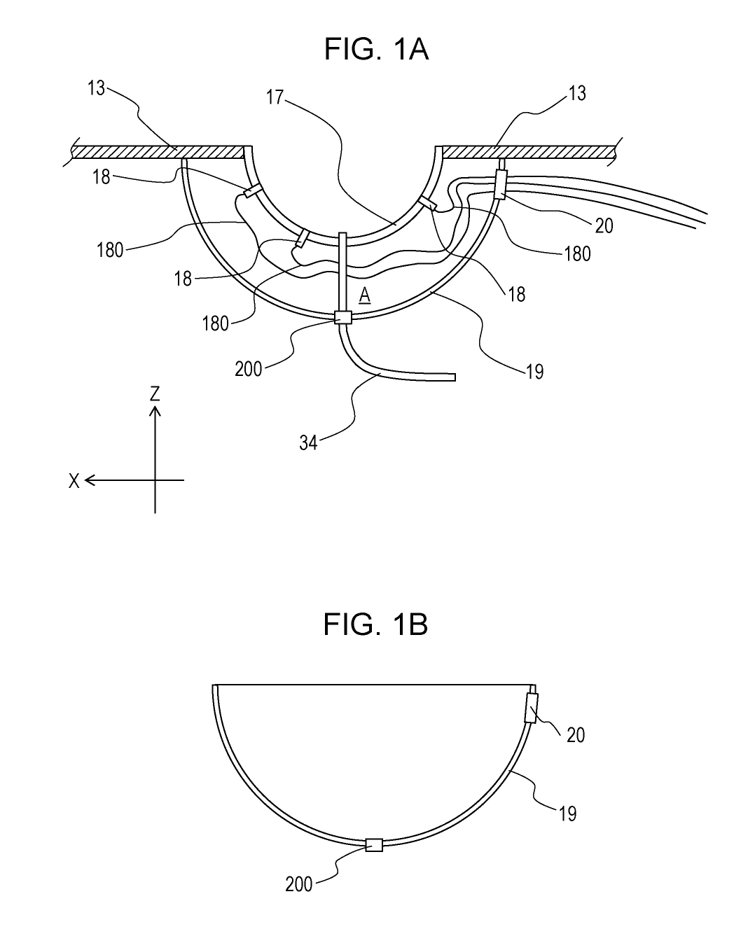

FIGS. 1A and 1B illustrate the structure of a breast examination section of a photoacoustic imaging apparatus according to a first embodiment of the present invention. FIG. 1A is a schematic sectional view taken along line IA-IA in FIG. 15. The left-right and top-bottom directions in FIG. 1A are the X-axis and Z-axis, respectively, and the direction perpendicular to the plane of FIG. 1A is the Y-axis. A transducer array 17 is structured such that a desired number of transducers 18 (for example, piezoelectric elements) are arranged spherically so as to be directed toward the center of a spherical surface. A cover member 19, which has a downwardly recessed portion, forms a housing space A, which covers a side opposite to a reception surface side of the transducer array 17 and accommodates the transducers 18, together with the transducer array 17 and a vessel 13. Outputs from the transducers 18 are transmitted through signal lines 180, which extend from the housing space A to the outside through a waterproof member 20 (first waterproof member) provided on the cover member 19. An optical waveguide 34, such as an optical fiber, guides pulsed light emitted from a light source to the transducer array 17. The optical waveguide 34 includes a water-repellent-finished light-emitting surface. The optical waveguide 34 extends from the isolated space to the outside through a waterproof member 200 provided on the cover member 19.

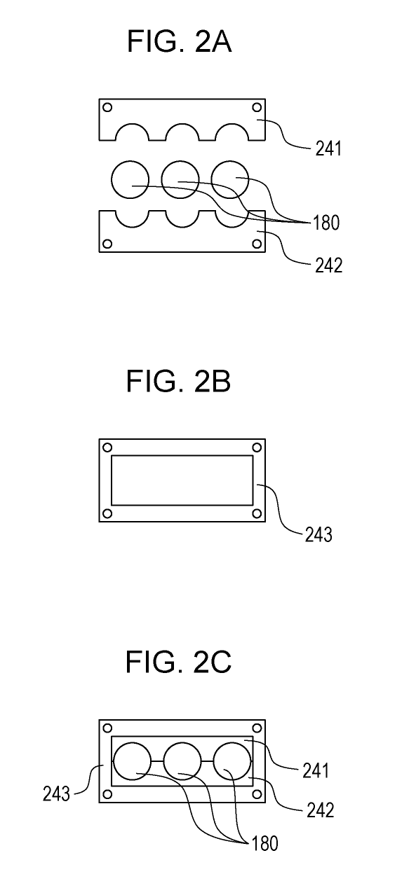

FIGS. 2A to 2C are schematic diagrams illustrating the waterproof member 20 provided on the cover member 19 viewed from the outside of the cover member 19, that is, from a side opposite to the side at which reception surfaces of the transducers 18 are arranged. Sleeves 241 and 242 are made of an elastic material, such as rubber, and semicircular recesses having a radius smaller than that of the signal lines 180 are formed therein. A frame 243 is made of, for example, a metal or a plastic, and has attachment screw holes formed therein, as illustrated in FIG. 2B. As illustrated in FIG. 2A, the signal lines 180 are sandwiched between the sleeves 241 and 242, and the frame 243 is placed on the sleeves 241 and 242 and fixed to the cover member 19 by inserting screws into holes in the cover member 19. Thus, the waterproof member 20 provided on the cover member 19 is completed (see FIG. 2C).

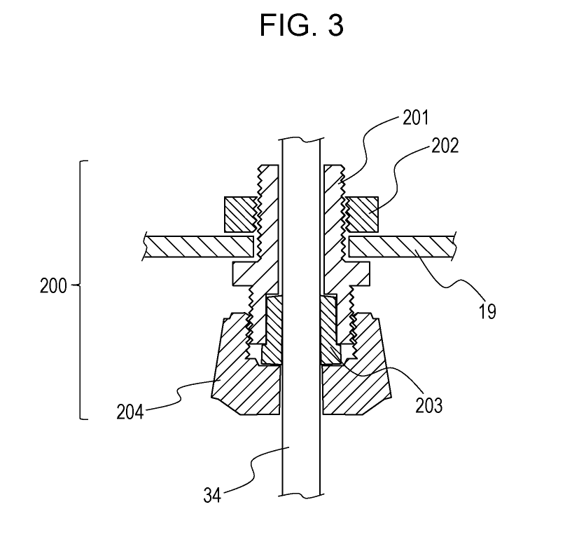

FIG. 3 is a schematic diagram illustrating the structure of the waterproof member 200 provided on the cover member 19. A cable ground body 201 is fixed to the cover member 19 with a lock nut 202. In the case where the cover member 19 has an irregular surface, a rubber packing (not shown) is disposed between the cable ground body 201 and the lock nut 202. The optical waveguide 34 is inserted through a rubber bush 203, the cable ground body 201, and a cap 204, and then the cap 204 is tightened, so that the optical waveguide 34 is fixed to the cover member 19. Thus, a waterproof member is provided.

The waterproof member 20 may have the same structure as that of the waterproof member 200. Alternatively, the waterproof members 20 and 200 may be formed by placing a grommet (rubber bush), waterproof silicon, or the like between the cover member 19 and each signal line 180 or between the cover member 19 and the optical waveguide 34.

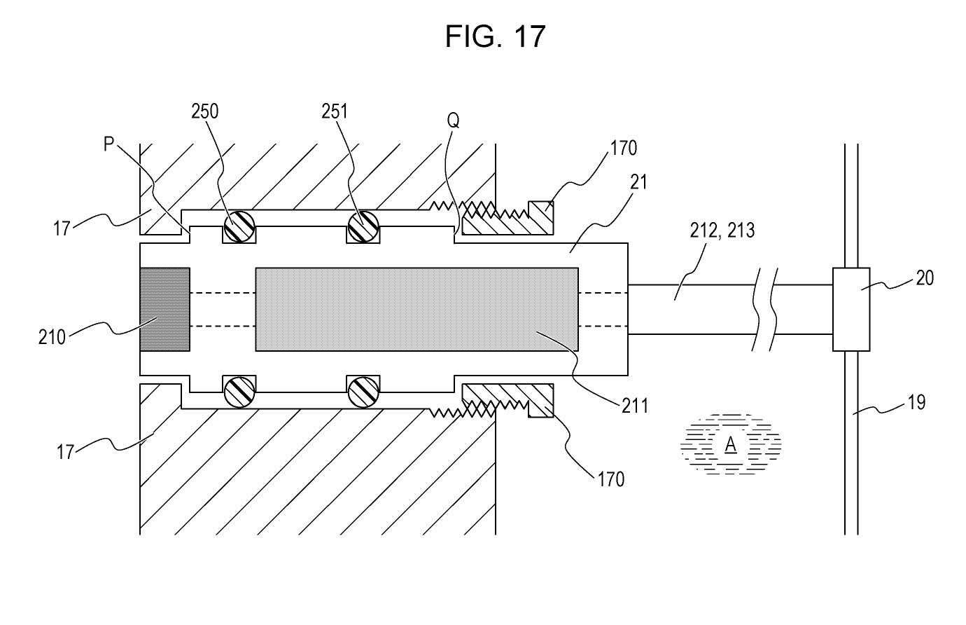

FIG. 1B illustrates the cover member 19 in FIG. 1A. The cover member 19 is fixed to the vessel 13 with screws, and is capable of being easily attached to and removed from the vessel 13 for, for example, maintenance of the transducers 18. A sealing member is disposed between the vessel 13 and the cover member 19. The transducers 18 have water-repellent-finished reception surfaces. The housing space A may have an internal waterproof structure in which an O-ring seal or the like is provided between each transducer 18 and the vessel 13. As illustrated in FIG. 17, a plurality of O-ring seals may be arranged along the thickness direction of a wall of the vessel 13 to improve the waterproof performance. With this structure, the transducers 18 can be easily removed from the vessel 13, and the maintenance can be facilitated. In the present embodiment, the transducers 18 are provided on a cup-shaped portion of the wall of the vessel 13. The space between the cover member 19 and each signal line 180 may also be water-repellent-finished in a similar manner by placing a sealing member, such as an O-ring seal, therein. Each transducer 18 may instead be bonded to the cup portion of the vessel 13 and water-repellent-finished by using an adhesive or a caulking material. The housing space A, which is surrounded by the transducer array 17, the vessel 13, and the cover member 19 and which accommodates the transducers 18, may be filled with, for example, dry air or nitrogen. In the case where water is used as the impedance matching liquid, the water may spill over from the vessel 13 when the transducer array 17 is moved to a desired position. Even in that case, the water does not adhere to the transducers 18 or the signal lines 180 and an increase in humidity does not occur. Accordingly, a change in signal intensity, which occurs when the impedance between the outputs of the transducers 18 is reduced as a result of an adhesion of the water thereto, and malfunction due to corrosion or the like can be avoided.

Also when a liquid other than water, such as castor oil, is used as the impedance matching liquid, dew condensation, which occurs when the temperature of the impedance matching liquid is lower than that of the apparatus, can be prevented. More specifically, even when the temperature on the inner side of the transducer array 17 suddenly decreases, the humidity in the housing space A is kept low, so that dew condensation does not occur in the housing space A. As a result, an adhesion of water to the transducers 18 or the signal lines 180 or an increase in humidity does not occur. A similar effect can also be obtained when the transducer array 17 is filled with water at a low temperature to clean the transducer array 17.

According to the present embodiment, the number of portions to be waterproofed is smaller than that in the case where portions through which the signal lines 180, which transmit the outputs from the transducers 18, extend to the outside are waterproofed. Accordingly, the assembly can be facilitated and the costs can be reduced.

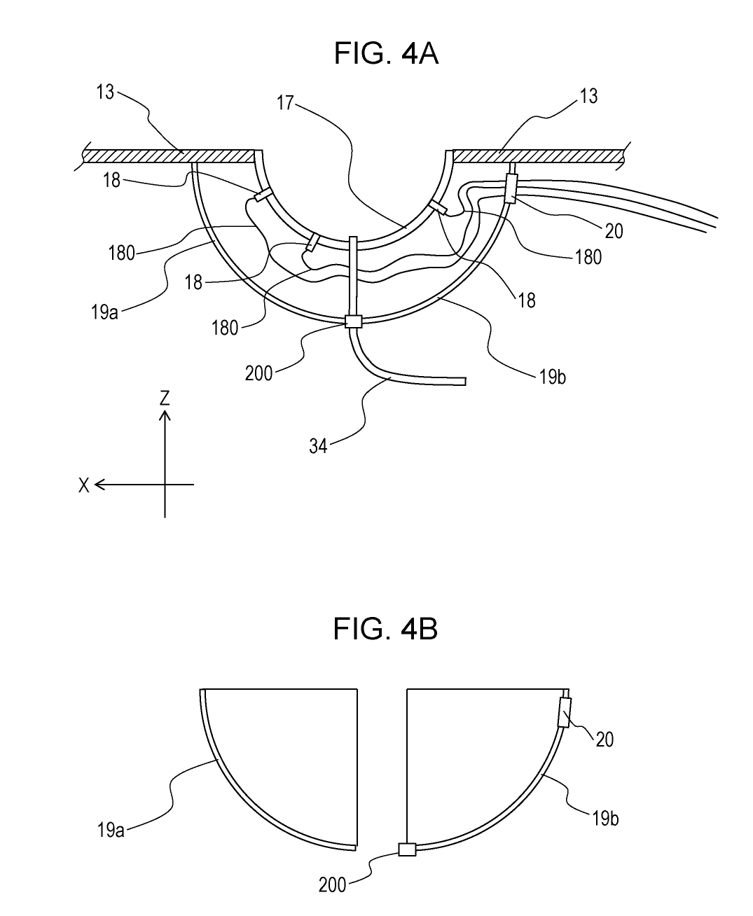

FIGS. 4A and 4B illustrate a modification of the present embodiment. In FIGS. 4A and 4B, cover pieces 19a and 19b form a housing space, which covers a side opposite to a reception surface side of the transducer array 17, together with the transducer array 17 and a vessel 13. The only difference from the structure illustrated in FIGS. 1A and 1B is that the cover member 19 can be divided into two pieces. Sealing members (not shown) are provided between the vessel 13 and the cover pieces 19a and 19b and between the cover piece 19a and the cover piece 19b. As illustrated in FIGS. 4A and 4B, the cover pieces 19a and 19b are separate components. Therefore, maintenance of transducers 18, for example, may be performed by removing only one cover piece 19a from the vessel 13. In this case, since the size of each cover piece is small, the work efficiency is increased. Even when both of the cover pieces need to be removed, the work efficiency is increased since the small cover pieces may be easily removed successively. These effects can be enhanced by increasing the number of pieces into which the cover member is divided. However, in such a case, the number of components for, for example, screw-fastening the cover pieces and the number of sealing members between the cover pieces increases. Therefore, when the number of cover pieces is simply increased, the structure will be complex and the costs will be increased. For this reason, the number of pieces into which the cover member is divided may be set to 2, 4, or 6 at a maximum. The cover member may also be divided in the Z direction. Moreover, the cover member may be divided into pieces at a location where an optical waveguide 34 is provided. In such a case, the cover pieces may be easily attached and removed without removing the optical waveguide 34 from the transducer array 17.

### Second Embodiment

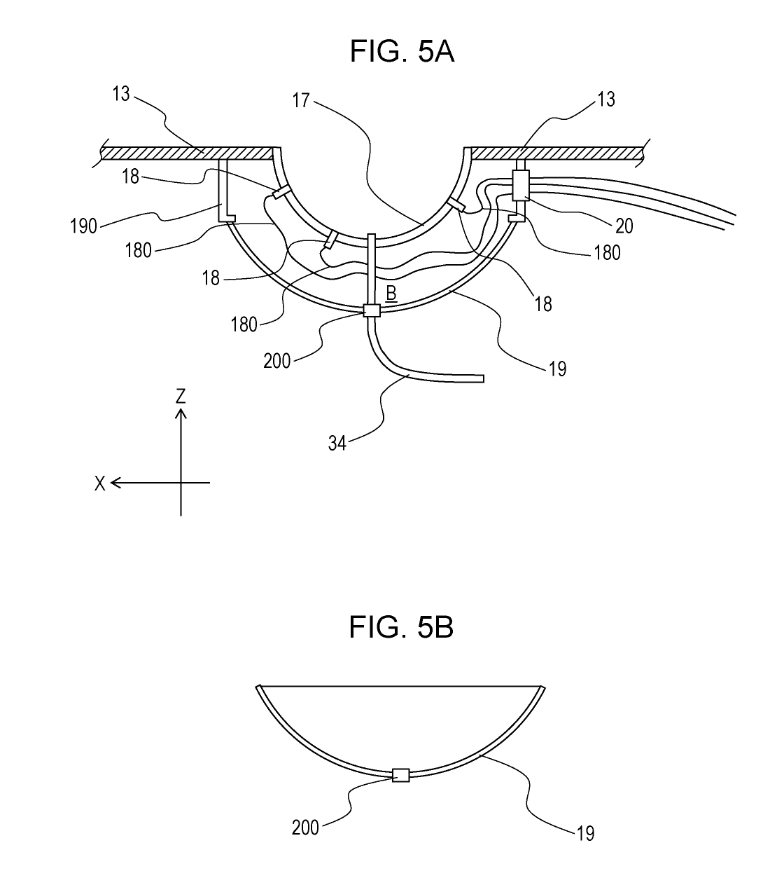

FIGS. 5A and 5B illustrate the structure of a breast examination section of a photoacoustic imaging apparatus according to a second embodiment of the present invention. FIG. 5A is a schematic sectional view taken along line VA-VA in FIG. 15. The left-right and top-bottom directions in FIG. 5A are the X-axis and Z-axis, respectively, and the direction perpendicular to the plane of FIG. 5A is the Y-axis.

A transducer array 17, a vessel 13, a cover member 19, and a connection member 190 form a housing space B, which covers a side opposite to a reception surface side of the transducer array 17 and which accommodates a plurality of transducers 18. The structure illustrated in FIGS. 5A and 5B differs from the structure illustrated in FIGS. 1A and 1B in that the cover member 19 illustrated in FIGS. 1A and 1B is replaced by two components, which are the connection member 190 and the cover member 19. The cover member 19 is attached to the connection member 190 with screws or the like with a sealing member interposed therebetween. The connection member 190 is fixed to the vessel 13. The connection member 190 may instead be fixed to the transducer array 17. Even in such a case, the connection member 190 forms the housing space that covers the side opposite to a reception surface side of the transducer array 17 together with the transducer array 17 and the cover member 19. In this case, the transducer array 17 may have the shape of a straw hat, and the connection member 190 may be fixed to a brim portion of the transducer array 17.

The present embodiment differs from the first embodiment in that the connection member 190 which connects the vessel 13 to the cover member 19 is provided. In the first embodiment, the outputs from the transducers 18 are transmitted through the signal lines 180 which extend from the housing space A accommodating the transducers 18 to the outside through the waterproof member 20 provided on the cover member 19. In this structure, the cover member 19 cannot be attached or removed without removing the signal lines 180 from the waterproof member 20 provided on the cover member 19. According to the present embodiment, signal lines 180 extend from the housing space B accommodating the transducers 18 to the outside through a waterproof member 20 mounted on the connection member 190. Therefore, the cover member 19 can be easily attached and removed without removing the signal lines 180 from the waterproof member 20.

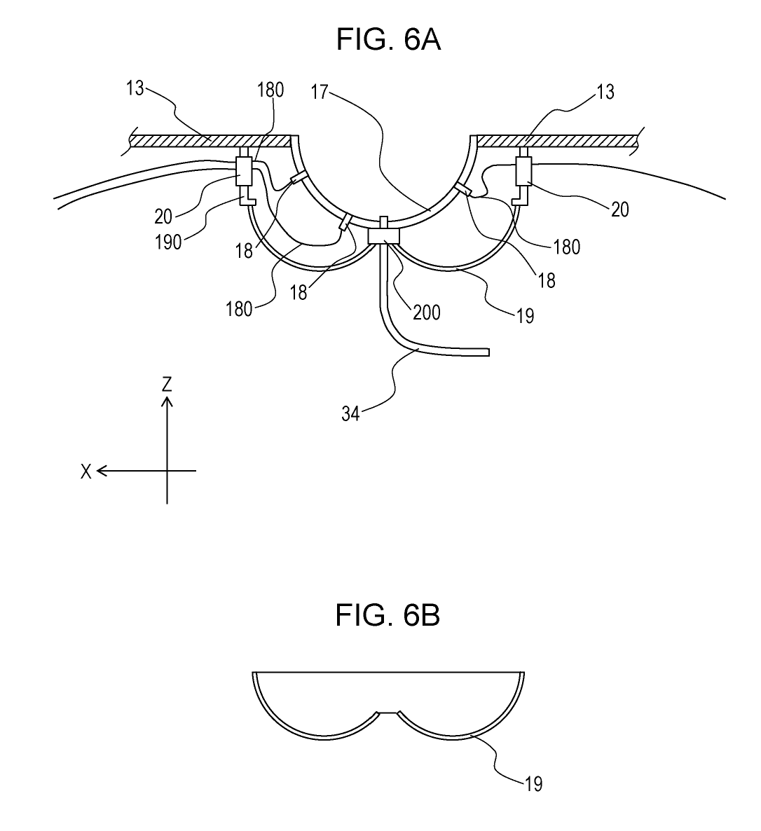

FIGS. 6A and 6B illustrate a modification of the present embodiment. In FIGS. 6A and 6B, an optical waveguide 34 extends to the outside through a waterproof member 200 provided on a cover member 19. The waterproof member 200 provided on the cover member 19 is fixed to a transducer array 17. The cover member 19 is attached to the waterproof member 200, which is fixed to the transducer array 17, with screws or the like with a sealing member (not shown) interposed therebetween. The structure illustrated in FIGS. 6A and 6B differs from the structure illustrated in FIGS. 5A and 5B in that the waterproof member 200 for the optical waveguide 34 is fixed to the transducer array 17. With this structure, the cover member 19 can be attached and removed by removing the optical waveguide 34 from the waterproof member 200. Therefore, the cover member 19 can be more easily attached and removed. The cover member 19 is shaped as illustrated in FIG. 6B. Owing to the structure illustrated in FIGS. 6A and 6B, the cover member 19 can be more easily attached and removed than in the first embodiment. To further facilitate the attachment and removal of the cover member 19, although not illustrated, the cover member 19 shown in FIGS. 6A and 6B may be divided into pieces at the location of the waterproof member 200 for the optical waveguide 34, as in the structure illustrated in FIGS. 4A and 4B. When the cover member is divided, the cover member can be attached and removed without removing the optical waveguide 34 from the waterproof member 200. Therefore, the attachment and removal of the cover member are further facilitated.

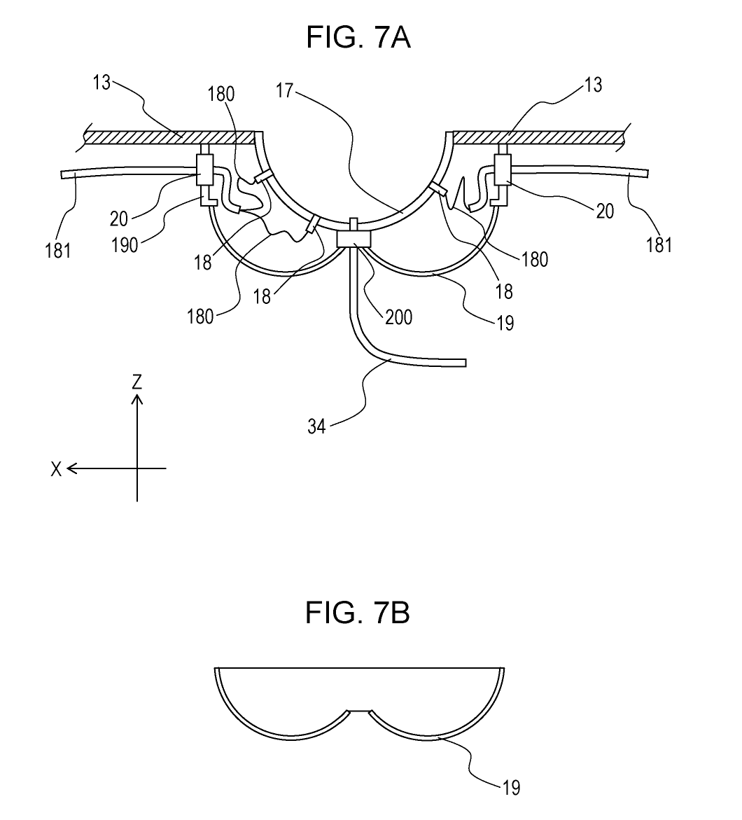

FIGS. 7A and 7B illustrate another modification of the present embodiment. Signal lines 180 that transmit the outputs from transducers 18 are bundled together into cables 181. The cables 181 have a structure in which a plurality of thin coaxial wires are bundled together and covered with a sheath. When the signal lines 180 that transmit the outputs are bundled together into a smaller number of cables 181, the structure of waterproof members 20 can be simplified. The cables 181 may also be applied to the other embodiments. As in the structure illustrated in FIGS. 4A and 4B, a cover member 19 may be divided into pieces at the location of a waterproof member 200 for an optical waveguide 34. When the cover member 19 is divided in this manner, the cover member 19 can be attached and removed without removing the optical waveguide 34 from the waterproof member 200.

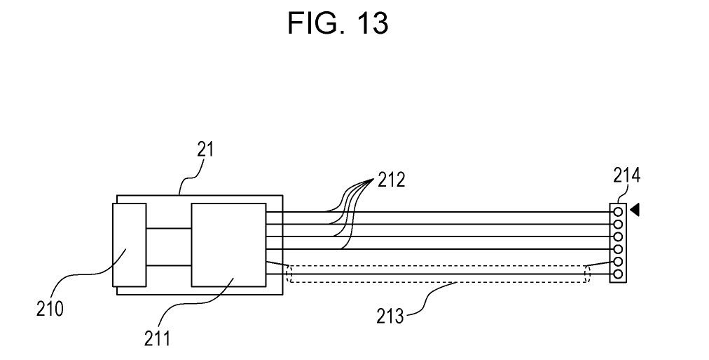

A photoacoustic imaging apparatus including capacitive micromachined ultrasonic transducer (CMUT) probes as capacitive micromachined ultrasonic transducer (CMUT) transducers will now be described. FIG. 13 is a block diagram illustrating the structure of a CMUT probe 21. A CMUT element 210 has a vibrating membrane that is extremely lighter and softer than that in a conventional transducer including a piezoelectric element. Therefore, a wide band signal can be received. The structure of the CMUT element 210 is described in detail in Japanese Patent Laid-Open No. 2009-165931. A current-voltage conversion circuit 211 converts a change in the capacitance of the CMUT element 210 into a voltage. Power supply lines 212 supply a bias voltage to the power supply of the current-voltage conversion circuit 211 and the CMUT element 210. A signal line 213 is, for example, a wire including a coaxial cable, and outputs the voltage into which the change in the capacitance of the CMUT element 210 is converted by the current-voltage conversion circuit 211. A connector 214 has pin 1 at the position indicated by the triangular mark. The CMUT probe 21 includes the CMUT element 210 and the current-voltage conversion circuit 211, and is mounted in a housing (not shown). As described below, the CMUT probe 21 is connected to a relay board by the power supply lines 212, the signal line 213, and the connector 214.

The CMUT probe 21 is configured such that the CMUT element 210 and the current-voltage conversion circuit 211 are located near each other, so that stray capacitance is reduced. Accordingly, a satisfactory SNR and a relatively large signal amplitude are obtained over a wide range. In other words, the CMUT element 210 and the current-voltage conversion circuit 211 cannot be easily arranged far from each other. Therefore, the CMUT probe 21 can be effectively used in a transducer array in which transducers are separated from each other with relatively large intervals (5 mm or more) therebetween. As described above, in a photoacoustic imaging apparatus according to the present invention including the transducer array 17 in which the transducers are arranged spherically so as to surround the subject, when 256 transducers are to be mounted, the transducers need to be arranged at a pith of about 23 mm. In such a case, the CMUT probe 21 including the CMUT element 210 and the current-voltage conversion circuit 211 is appropriate.

An embodiment in which a connecting section between a cup portion of a vessel 13 and a CMUT probe 21 is provided with an internal waterproof structure will be described with reference to FIG. 17.

FIG. 17 is a schematic sectional view illustrating the internal waterproof structure between the cup portion of the vessel 13 and the CMUT probe 21. Referring to FIG. 17, a hollow bolt 170 having an external thread meshes with an internal thread on the inner surface of a CMUT-probe attachment hole formed in the cup portion of the vessel 13.

Sealing members 250 and 251 are arranged between the vessel 13 and the CMUT probe 21 to ensure liquid tightness. In the present embodiment, O-ring seals made of a silicone rubber are used. The sealing members are selected in consideration of the chemical stability against impedance matching liquid, durability against swelling, and elastic characteristics. For example, silicone rubber, Teflon-based rubber (Teflon is a registered trademark), or the like may be used. The sealing members are not limited to O-ring seals as long as the shape thereof matches the shape of the CMUT probe 21 and the shape of the attachment portion of the vessel 13.

A CMUT element 210 is mounted on a reception surface of the CMUT probe 21 so that the CMUT element 210 is directed toward the center of a transducer array 17. Power supply lines 212 and a signal line 213 are inserted through the hole in the hollow bolt 170, and the CMUT probe 21 is inserted into the CMUT-probe attachment hole formed in the cup portion of the vessel 13. The hollow bolt 170 is turned while the external thread on the hollow bolt 170 meshes with the internal thread on the inner surface of the CMUT-probe attachment hole, so that the CMUT probe 21 is reliably fixed to the cup portion of the vessel 13. At this time, the hollow bolt 170 presses a pressing surface Q of a housing of the CMUT probe 21, so that the CMUT probe 21 is positioned with respect to the cup portion of the vessel 13 at an abutting surface P. Owing to this positioning mechanism, unlike the case in which the positioning mechanism is not provided, that is, the case in which the abutting surface P is not provided, the sealing members 250 and 251 do not receive a pressing force applied by the hollow bolt 170. Therefore, deformation, such as flattening, of the sealing members 250 and 251 does not occur. The CMUT probe 21 secured to the vessel 17 with sealing members 250 and 251 and the cover member 19 with the water proof member 20(a-e) forms a sealed space as the housing space A. The waterproof structure includes an internal waterproof structure having a plurality of ring seals (sealing members) 250 and 251 arranged along a thickness direction of the wall of the vessel 13 between the vessel 13 and each one of transducers 21 such that the internal waterproof structure seals the vessel 13 configured to prevent from being occurred a leakage into the housing space A. As a result, the waterproof function between the inside and outside of the cup portion of the vessel 13 can be enhanced. Unlike other waterproof members, in the internal waterproof structure between the cup portion of the vessel 13 and the CMUT probe 21, the housing of the CMUT probe 21 is in direct contact with the impedance matching material, such as water. Therefore, a waterproof member having at least two stages may be used.

The above-described internal waterproof structure for the CMUT probe 21 may be applied to the above-described first and second embodiments, and may also be applied to embodiments described below. Although an internal waterproof structure between the cup portion of the vessel 13 and the CMUT probe 21 is described, the internal waterproof structure may also be applied to other ultrasonic transducers, such as piezoelectric transducers.

### Third Embodiment

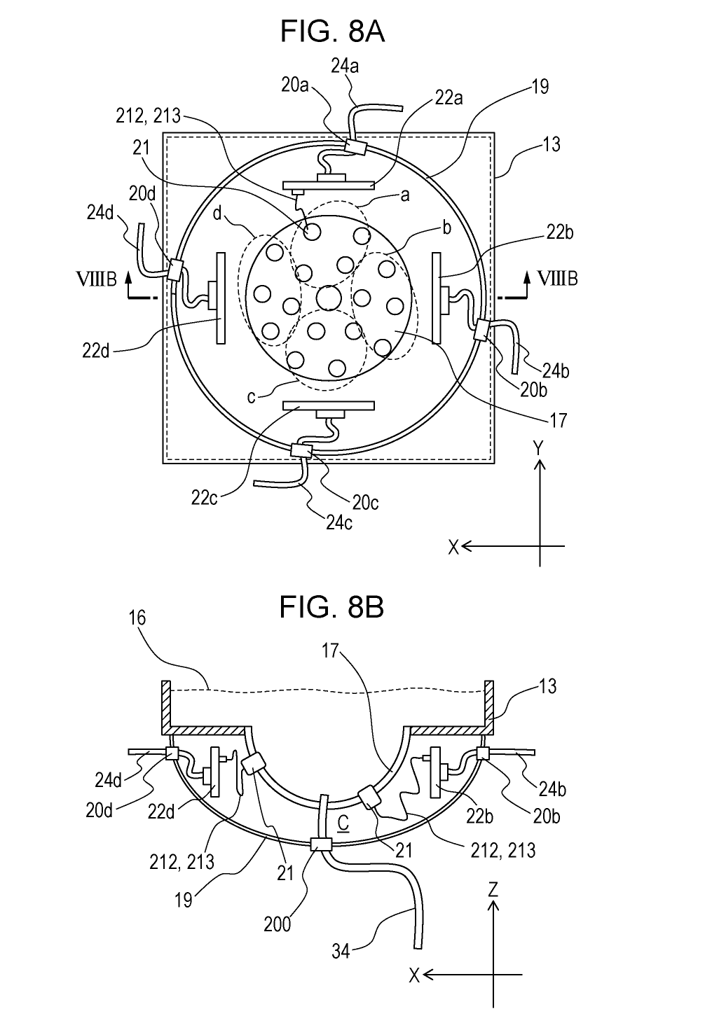

FIGS. 8A and 8B illustrate the structure of a breast examination section of a photoacoustic imaging apparatus according to a third embodiment of the present invention. FIG. 8A is a schematic diagram illustrating a transducer array 17 viewed from below a support base 10 that supports an examinee. The left-right and top-bottom directions in FIG. 8A are the X-axis and Y-axis, respectively, and the direction perpendicular to the plane of FIG. 8A is the Z-axis.

The transducer array 17 is structured such that a desired number of CMUT probes 21 are arranged spherically so as to be directed toward the center of a spherical surface. The transducer array 17 and relay boards 22a, 22b, 22c, and 22d are fixed to a vessel 13. For example, the CMUT probes 21 in the region indicated by ‘a’ are connected to the relay board 22a by power supply lines 212 and a signal line 213. The CMUT probes 21 in the regions indicated by ‘b’, ‘c’, and ‘d’ are connected in a similar manner. Although only the power supply lines 212 and the signal line 213 for a single CMUT probe 21 is shown in FIG. 8A to avoid complexity, all of sixteen CMUT probes 21 are connected to their respective power supply lines 212 and signal lines 213. The power supply lines and signal lines are bundled together in the relay boards, and are connected to a power supply and a photoacoustic reception unit, which will be described below, in the form of power supply line cables and signal line cables. In FIG. 8A, these cables are simply shown as cables 24a, 24b, 24c, and 24d to avoid complexity. The cables 24a, 24b, 24c, and 24d extend from a housing space C, which accommodates the transducers, to the outside through waterproof members 20a, 20b, 20c, and 20d. The portions through which the signal lines, which transmit the outputs from the CMUT probes 21, extend to the outside are isolated from the outside by the transducer array 17, the vessel 13, and a cover member 19. The housing space C, which accommodates the transducers, is filled with, for example, dry air or nitrogen. As in the above-described case, the cover member 19 may be dividable into a plurality of pieces at, in particular, a location where an optical waveguide 34 is attached to a waterproof member 200. In such a case, the cover member can be easily attached and removed.

FIG. 8B is a schematic sectional view taken along line VIIIB-VIIIB in FIG. 8A. The left-right and top-bottom directions in FIG. 8B are the X-axis and Z-axis, respectively, and the direction perpendicular to the plane of FIG. 8B is the Y-axis. The relay boards 22b and 22d are fixed to the vessel 13 with attachment screws (not shown).

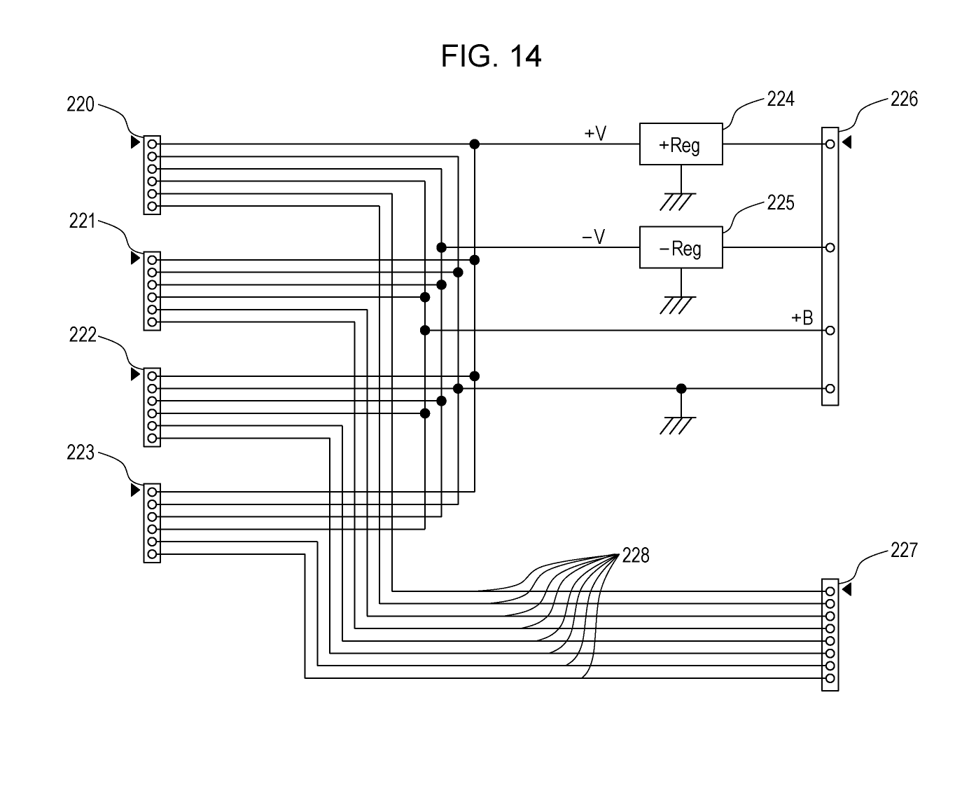

The relay boards 22a, 22b, 22c, and 22d will be described with reference to FIG. 14. The relay boards 22a, 22b, 22c, and 22d have the same circuit structure. Connectors 220, 221, 222, and 223 are connected to connectors 214 of the CMUT probes 21. In the present embodiment, four relay boards are provided for sixteen CMUT probes 21. Therefore, each relay board is connected to four connectors. Signal lines 228, to which signals from the CMUT probes 21 are input, are connected to a signal connector 227, and are output through a signal line cable. Power supply lines of the CMUT probes 21 correspond to pins 1 to 4 of the connectors 220, 221, 222, and 223. These power supply lines are bundled together and connected to a power supply connector 226, and are output in the form of a power supply line cable.

In the case where the CMUT probes are used, a high-density wiring pattern is printed on each relay board, and a high voltage for a bias power supply is applied to the wires. Therefore, a large voltage difference is generated between the wiring pattern for the bias power supply of each relay board and a GND pattern. However, since the relay boards are disposed in the housing space C that accommodates the transducers, the occurrence of ion migration between the patterns in a high electrical field can be suppressed. Thus, when the CMUT probes are used, unlike the case where piezoelectric elements are used, ion migration can be suppressed.

The wires that extend from the power supply connector 226 and the signal connector 227 of each relay board to the power supply and the photoacoustic reception unit, which will be described below, are relatively long. Therefore, a voltage drop occurs between the power supply connector 226 and the power supply. The voltage drop occurs because the current is increased since the power supply lines of the CMUT probes 21 are bundled together and because the electrical resistance is increased since the wire that extends from the power supply connector 226 to the power supply, which will be described below, is long. Therefore, local regulators 224 and 225 are additionally provided in the wires for transmitting electricity to the CMUT probes 21, so that a stabilized voltage can be supplied to the CMUT probes 21. The local regulators 224 and 225 may be, for example, integrated circuits called three-terminal regulator ICs. Alternatively, stabilizing power supply circuits composed of discrete components may be used. Since substantially no current flow occurs in the 3-pin bias power supply of the power supply connector 226, the above-described voltage drop hardly occurs. Therefore, there is no need to mount a local regulator. To reduce the noise caused because the wire from the power supply connector 226 to the power supply, which will be described below, is long, although not illustrated, noise filters may be additionally provided in the power supply lines of each relay board to reduce the power supply noise.

In the present embodiment, the relay boards are disposed in the housing space C that accommodates the transducers. Therefore, a change in signal intensity, which occurs when the impedance is reduced as a result of an adhesion of water to the outputs of the CMUT probes 21, and malfunction due to corrosion or the like can be avoided. In addition, since ion migration in the bias circuit can be suppressed, the reception performance can be increased. In addition, with the structure according to the present invention, the number of portions to be waterproofed is smaller than that in the case where the relay boards 22a, 22b, 22c, and 22d and portions through which the signal lines, which transmit the outputs from the CMUT probes 21, extend to the outside are waterproofed. Accordingly, the assembly can be facilitated and the costs can be reduced. In addition, unlike the second embodiment, the wires can be bundled together into cables at the relay boards, and therefore the mounting process can be facilitated. The relay boards may also be used in the first embodiment and the second embodiment, in which the piezoelectric elements are used as the transducers 18, to bundle the wires into cables.

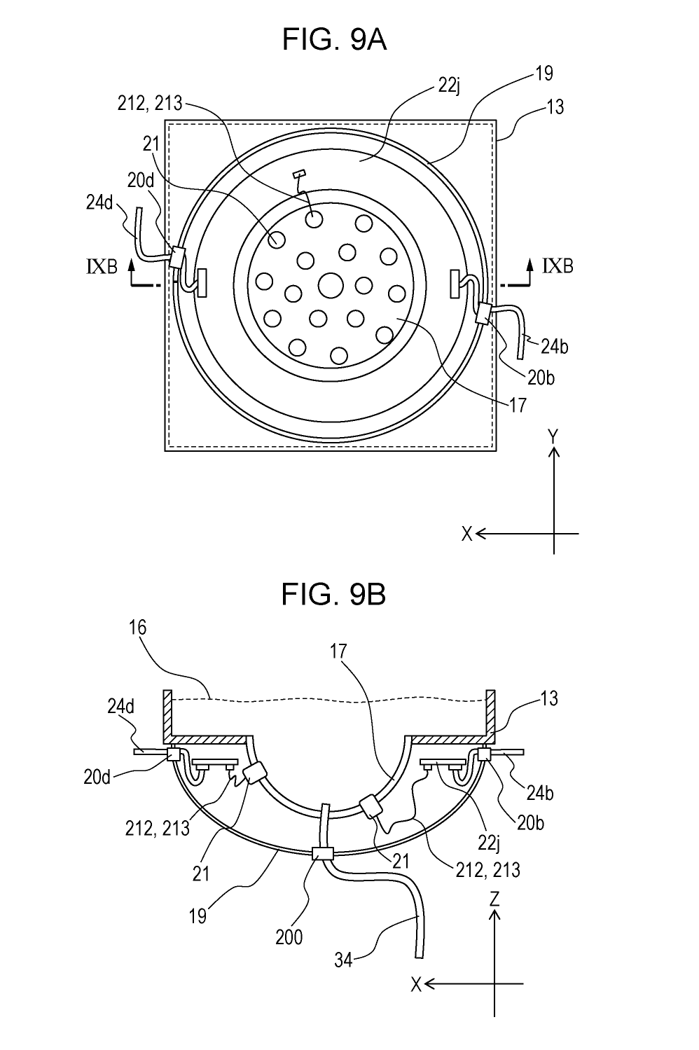

FIGS. 9A and 9B illustrate a modification of the present embodiment. FIG. 9A is a schematic diagram illustrating a transducer array 17 included in a photoacoustic imaging apparatus of this modification, viewed from below a support base 10 that supports an examinee. FIG. 9B is a schematic sectional view taken along line IXB-IXB in FIG. 9A. A relay board 22j has the shape of an annular disc that is substantially concentric to the transducer array 17. CMUT probes 21 are connected to the relay board 22j by power supply lines 212 and signal lines 213. The configurations of wires and cables on the relay board and waterproof members 20b and 20d are the same as those in the above-described embodiment.

The relay board 22j is fixed to a vessel 13 with screws (not shown) such that the center of the relay board 22j substantially aligns with the center of the transducer array 17. Unlike the above-described embodiment, the number of relay boards is one. However, in the case where a large number of CMUT probes 21 are mounted on the transducer array 17, a physically single print board may be divided into a plurality of blocks in terms of electrical circuits. More specifically, the relay board 22j may be designed such that the relay board 22j has the shape of a single annular disc but includes, for example, four independent electrical circuits as in the above-described embodiment. In this case, the current that flows through the power supply lines between the relay board 22j and a power supply 7 may be distributed, and a voltage drop can be reduced accordingly. In addition, the size of radiators for the heat generated by the local regulators can be reduced by using separate regulator ICs.

In this modification, since the number of relay boards is one, the assembly and maintenance of the apparatus can be further facilitated.

### Fourth Embodiment

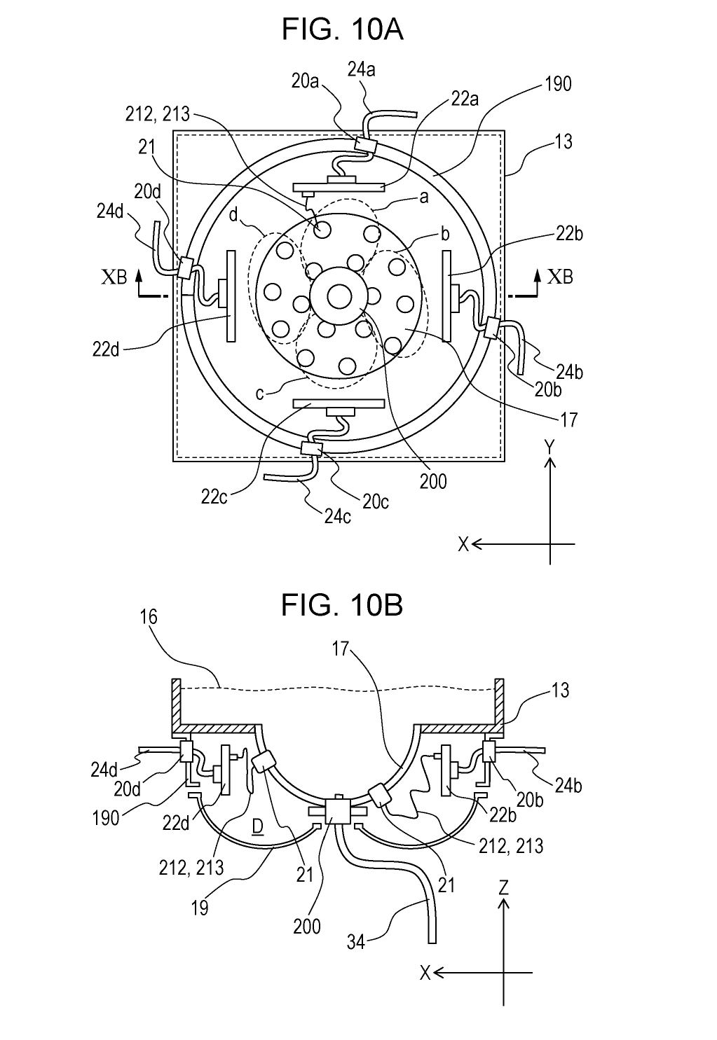

A fourth embodiment of the present invention will be described with reference to FIGS. 10A and 10B. The structure of the present embodiment is obtained by applying the second embodiment to the third embodiment. FIG. 10A is a schematic diagram illustrating a transducer array 17 included in a photoacoustic imaging apparatus viewed from below a support base 10 that supports an examinee. FIG. 10B is a schematic sectional view taken along line XB-XB in FIG. 9A.

A cover member 19 is attached to a connection member 190 that is fixed to the transducer array 17. Cables 24a, 24b, 24c, and 24d extend from a housing space D, which accommodates a plurality of transducers, to the outside through waterproof members 20a, 20b, 20c, and 20d mounted on the connection member 190. Accordingly, the cover member 19 can be attached and removed without removing the cables 24a, 24b, 24c, and 24d from the waterproof members 20a, 20b, 20c, and 20d.

The fourth embodiment differs from the third embodiment in that the waterproof members 20a, 20b, 20c, and 20d are mounted on the member 190 fixed to the transducer array 17, and the cover member 19 can be attached and removed without removing the cables 24a, 24b, 24c, and 24d from the waterproof members 20a, 20b, 20c, and 20d. Thus, the attachment and removal of the cover member 19 are facilitated. A waterproof member 200, through which an optical waveguide 34 extends from the housing space D that accommodates the transducers to the outside, is fixed to the transducer array 17. Therefore, the cover member 19 can be easily attached and removed by removing the optical waveguide 34 from the waterproof member 200 that is fixed to the transducer array 17. In the case where the cover member 19 is dividable into pieces at the location of the waterproof member 200, the cover member 19 can be attached and removed without removing the optical waveguide 34 from the waterproof member 200. In such a case, the cover member can be more easily attached and removed, and maintenance can be facilitated.

According to the present embodiment, the number of portions to be moisture-proofed and/or waterproofed is smaller than that in the case where the relay boards 22a, 22b, 22c, 22d, and 22j and portions through which signal lines that transmit outputs from the CMUT probes 21 extend to the outside are waterproofed. Therefore, the assembly can be facilitated and the costs can be reduced. In addition, since the waterproof members 20a, 20b, 20c, and 20d are provided on the connection member 190, the cover member 19 can be easily attached and removed and maintenance can be facilitated.

### Fifth Embodiment

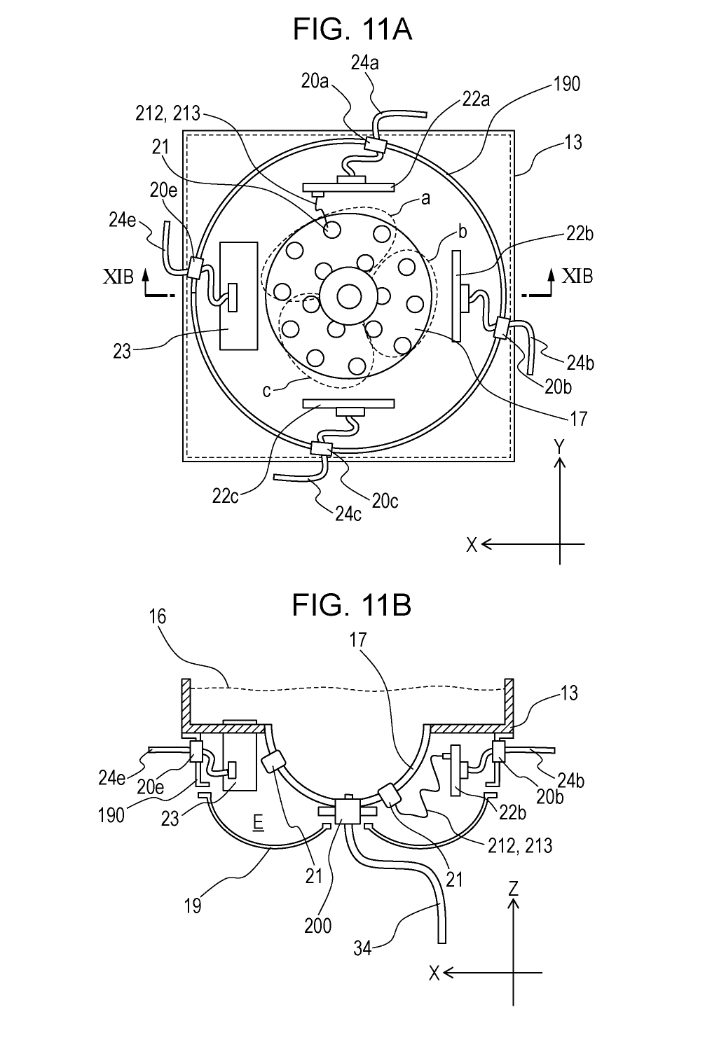

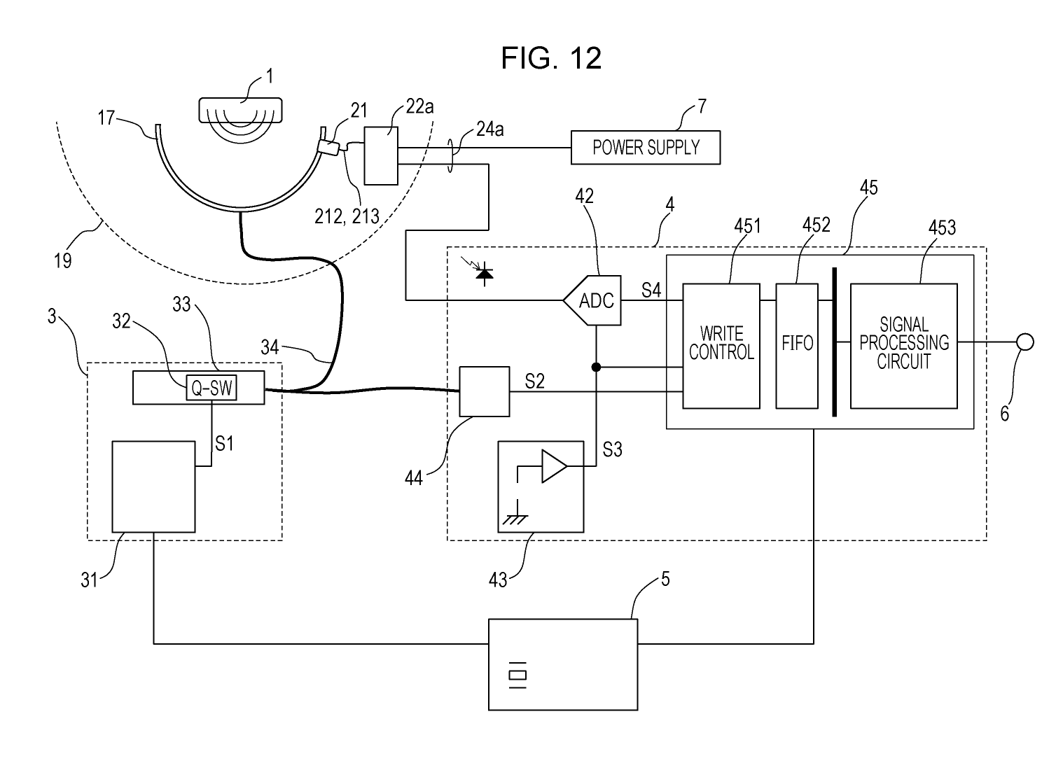

A fifth embodiment is similar to the fourth embodiment except an additional electrical unit is mounted in a housing space that accommodates a plurality of transducers. FIGS. 11A and 11B illustrate the structure of a breast examination section of a photoacoustic imaging apparatus according to the fifth embodiment. FIG. 11A illustrates a transducer array 17 viewed from below a support base 10 that supports an examinee. FIG. 11B is a schematic sectional view taken along line XIB-XIB in FIG. 11A.

Although a single cable 24e is shown in FIGS. 11A and 11B, cables for power supply and signal output may instead be separately provided. The cable 24e extends from a housing space E, which accommodates a plurality of transducers, to the outside through a waterproof member 20e. An ultrasonic transmission-reception unit 23 is fixed to a vessel 13. A transmission-reception surface of the ultrasonic transmission-reception unit 23 is water-repellent-finished, and is directed toward the subject (Z direction). The ultrasonic transmission-reception unit 23 and the vessel 13 are bonded together with an adhesive or a caulking material, and are water-repellent-finished. When the ultrasonic transmission-reception unit 23 is mounted on the vessel 13, not only an image based on photoacoustic waves but also an image based on the reflection of ultrasonic waves can be obtained by using a single apparatus. Therefore, the diagnostic accuracy can be increased.

According to the present embodiment, the ultrasonic transmission-reception unit 23, which needs to be waterproofed, may be mounted in the housing space E, which accommodates the transducers, in addition to the portions through which signal lines that transmit outputs from the CMUT probes 21 extend to the outside and the relay boards 22a, 22b, and 22c. Accordingly, the number of portions to be waterproofed is smaller than that in the case where the components are individually waterproofed.

In the present embodiment, the ultrasonic transmission-reception unit 23 is mounted in the housing space that accommodates the transducers. However, similar effects can also be obtained when other types of electronic units are mounted in the housing space. For example, similar effects can also be obtained when the electronic unit mounted in the housing space is, for example, an optical sensor that detects whether or not the breast, which is the subject, is held by the breast holder 12; a liquid surface sensor that determines whether or not the liquid surface 16 of the impedance matching liquid is at a normal level; a liquid temperature sensor that detects the temperature of the impedance matching liquid; or a monitor camera for monitoring the breast, which is the subject.

### Sixth Embodiment

A sixth embodiment of the present invention will now be described. The sixth embodiment may be applied to all of the apparatuses according to the first to fifth embodiments.

To maintain the state in which the humidity in the housing space that accommodates the transducers is low, a drying agent or the like is disposed in the space. The drying agent may be, for example, a physical drying agent having a porous structure, such as silica gel or zeolite. Moreover, the apparatus may be provided with a sensor that measures the humidity in the housing space that accommodates the transducers, and have a function of notifying the user that maintenance is required when the humidity increases.

The drying agent absorbs water, and the weight thereof increases accordingly. Therefore, the drying agent may be bonded to the inner surface of the cover member 19 or hung from the vessel 13 in the housing space that accommodates the transducers.

The cover member 19 may be made of a transparent material so that the inside of the housing space that accommodates the transducers can be easily observed. For example, a plastic material such as polycarbonate may be used. In particular, in the case where a drying agent or the like is disposed in the space, it is appropriate to form the cover member 19 by using, for example, a transparent resin material so that the inside of the space can be easily observed.

The transducer array 17, the vessel 13, the cover member 19, and the connection member 190 may all be formed of conductive members, so that the housing space that accommodates the transducers has a shielding effect against external electromagnetic waves. When the electromagnetic shielding effect is provided, noise applied to the transducers 18 owing to the external magnetic waves can be reduced. In the case where the cover member 19 is formed of a transparent plastic material, an ITO film may be formed by sputtering or an application method to impart conductivity.

The waterproof members may be composed of members called cable ground or cable entry systems. Alternatively, packings made of a silicone rubber or the like may be used.

In the apparatuses according to the first to fifth embodiments, a container composed of the transducer array 17 and the vessel 13 is filled with the impedance matching liquid, so that impedance matching with the breast, which is the subject, is achieved.

### Circuit Structure and Operation of Photoacoustic Imaging Apparatus

The circuit structure and the operation of a photoacoustic imaging apparatus according to the present embodiment will now be described with reference to FIG. 12. FIG. 12 illustrates the circuit structure of a photoacoustic imaging apparatus in which CMUT probes described in the third embodiment are used as transducers. In the case where a cover member 19 is conductive and grounded, the CMUT probes 21 and other components can be shielded. FIG. 12 only illustrates wires of a CMUT probe 21 and a relay board 22a for a single CMUT element to avoid complexity; however, other CMUT elements have a similar structure. Although multiple CMUT probes 21 are mounted on a transducer array 17, FIG. 12 only illustrates components for a single CMUT element to facilitate the description. Memories 452 provided for the respective CMUT elements are arranged in parallel.

Referring to FIG. 12, a laser light control circuit 31 included in a laser-pulse transmission unit 3 outputs an oscillation start signal S1 to a Q switch 32 in accordance with a laser emission command output by a system controller 5, which serves as a light emission timing controller. A laser device 33 included in the laser-pulse transmission unit 3 emits laser pulses at a timing of the oscillation start signal S1. The Q switch 32 may instead be an oscillation controller having another structure. For example, when a semiconductor laser is used, a modulation driver may be used instead of the Q switch 32 because the response speed of direct modulation is sufficiently high. In other swords, the modulation driver serves as the oscillation controller. The laser pulses are guided through a fiber 34, and a breast 1, which is a subject, is irradiated with the laser pulses. When the laser pulses reach the breast 1, photoacoustic waves are generated in accordance with the absorption efficiency of the breast 1. As described below, a photoacoustic reception unit 4 receives the photoacoustic waves and converts the photoacoustic waves into a tomographic image (image reconstruction process). The photoacoustic waves are input to CMUT elements and converted into analog electric signals (photoacoustic signals). The analog electric signal (photoacoustic signal) obtained by each CMUT probe 21 is converted into digital data (photoacoustic data) S4 by an AD converter 42. A sampling clock signal S3 input to the AD converter 42 is a stable reference clock that is generated by a reception reference clock circuit 43 and in which the amount of jitter is small. The writing timing at which the photoacoustic data S4 is written into the memory 452 is determined by a light reception trigger signal S2, which is output from a photodetector 44. A desired number of pieces of digital data (photoacoustic data) S4 obtained by the AD conversion after the determined writing timing are successively stored in the memory 452. In other words, from the time at which the light reception trigger signal S2 is input, a desired number of pieces of digital data (photoacoustic data) S4, which are obtained by the AD converter 42 at the timing of the sampling clock signal S3, are successively stored in the memory 452 by a writing control circuit 451 included in a signal processor 45. A signal processing circuit 453 reads the photoacoustic data corresponding to all of the CMUT elements from the respective memories, and performs signal processing (image reconstruction) to generate image data for visualizing the inside of the body based on the photoacoustic waves (tomographic image data). The image data is output from an output terminal 6. Although the output terminal 6 from which the reconstructed image data is output is described in the present embodiment, a network input/output terminal through which the reconstructed image data is stored in a memory (not shown) or a nonvolatile memory that simply stores the reconstructed image data, for example, may instead be provided. Although the signal processing circuit 453 illustrated in FIG. 12 is a hardware component that performs the process, the process may instead be performed by a software program. In particular, the image reconstruction can be performed in a relatively short time when a multi-core CPU, which has been recently developed, is used. In the case where a software program is used, a process of reading data from the memory 452 is performed. In this case, the laser-pulse emission timing may be determined so that the software processing load does not concentrate. The signal processing for obtaining a tomographic image (image reconstruction) is described in detail in Japanese Patent Laid-Open No. 2011-5042, and description thereof is thus omitted.

The electricity is supplied to the CMUT probes 21 from a power supply 7. The power supply 7 is arranged in, for example, the same rack as the photoacoustic reception unit 4. In the case where local regulators are mounted in relay boards 22a, 22b, 22c, and 22d, the power supply 7 outputs a voltage higher than a voltage obtained by adding a power supply voltage of the CMUT probes 21 to the sum of a voltage drop between the input and output of the local regulators and a voltage drop in the wires from the relay boards 22a, 22b, 22c, and 22d to the power supply 7. Although the above description is based on the assumption that the regulators are positive-voltage regulators, it also applies to the case where the regulators are negative-voltage regulators when absolute values are considered. Thus, by appropriately setting the voltage of the power supply 7, a desired voltage can be reliably applied to the CMUT probes 21.

As described above, an image based on optical ultrasonic waves can be obtained by the photoacoustic imaging apparatus having the structure illustrated in the electrical circuit block diagram of FIG. 12.

Although the CMUT probes 21 are used as the transducers in the structure illustrated in FIG. 12, an image based on optical ultrasonic waves can also be obtained in a similar manner when piezoelectric elements, for example, are used as the transducers. In such a case, the power supply 7 is not necessary, and the relay board 22a is not always necessary. The basic operation in the case where piezoelectric elements are used as the transducers is the same as that in the case where the CMUT probes 21 are used.

The present invention may also be applied to thermoacoustic scanner apparatuses that forms an image based on acoustic waves generated in response to a thermal expansion of a biological tissue when electromagnetic radiation pulses are applied. In such a case, the optical waveguide 34, such as an optical fiber, described in the above-described embodiments corresponds to a circular waveguide for emitting radiation pulses having a short emission time period transmitted from an RF pulse emission unit, such as a microwave generator, toward a transducer array. Alternatively, the optical waveguide 34 corresponds to a transmission line that guides RF pulses of microwaves to the circular waveguide. The present invention may be advantageously applied to apparatuses that receive acoustic waves generated in a biological tissue with a transducer array having a downwardly recessed portion along which transducers are arranged and form an image based on the acoustic waves generated in the biological tissue.

According to the present invention, the transducers can be waterproofed with a simple structure. As a result, the risk of malfunction of the transducers that occurs when water adheres to the transducers or signal lines or when the humidity in the housing space that accommodates the transducer array is increased can be reduced. In addition, the risk of reduction in reception performance of the transducers due to corrosion or ion migration can also be reduced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-249408 filed Dec. 9, 2014 and No. 2015-168285 filed Aug. 27, 2015, which are hereby incorporated by reference herein in their entirety.

What is claimed is:

1. An acoustic imaging apparatus comprising:
an image forming unit including a vessel that accommodates impedance matching liquid and a transducer array that is disposed on a wall of the vessel and on which a plurality of transducers are arranged, the transducers including reception surfaces that receive acoustic waves generated by a subject, the image forming unit forming an image based on outputs from the transducers; and
a cover member that covers the transducer array at a side opposite to a side at which the reception surfaces are arranged so that the vessel and the cover member form a housing space that accommodates the transducers, the cover member being connected to the vessel, the transducers being provided with signal lines through which electric signals based on the received acoustic waves are output,
wherein the signal lines extend from the housing space to the outside through a waterproof member.

2. The acoustic imaging apparatus according to claim 1, wherein a drying agent is disposed in the housing space that accommodates the transducers.

3. The acoustic imaging apparatus according to claim 1, wherein the reception surfaces of the transducers are water-repellent-finished.

4. The acoustic imaging apparatus according to claim 1, further comprising:
a light source that generates pulsed light,
wherein the transducer array detects photoacoustic waves generated in response to the pulsed light.

5. The acoustic imaging apparatus according to claim 4, further comprising:
an optical waveguide configured to guide the pulsed light generated by the light source to an inner side of the transducer array,
wherein the optical waveguide extends from the housing space that accommodates the transducers to the outside through a waveguide waterproof member provided on the cover member.

6. The acoustic imaging apparatus according to claim 5, wherein the cover member is dividable into a plurality of members, and the waveguide waterproof member is disposed at a location where the cover member is dividable.

7. The acoustic imaging apparatus according to claim 1, further comprising:
a support member that has an insertion opening into which the subject is inserted, the support member supporting an examinee from below,
wherein the transducer array is disposed below the insertion opening,
wherein the cover member is fixed to the support member with a fixing member, and
wherein the waterproof member is disposed on the fixing member.

8. The acoustic imaging apparatus according to claim 7, wherein the transducer array, the fixing member, and the cover member are conductive.

9. The acoustic imaging apparatus according to claim 1, wherein at least one selected from the group consisting of an ultrasonic transmission-reception unit, a liquid surface sensor, a liquid temperature sensor, and a monitor camera is disposed in the housing space that accommodates the transducers.

10. The acoustic imaging apparatus according to claim 1, wherein the waterproof structure includes an internal waterproof structure having a plurality of ring seals arranged along a thickness direction of the wall of the vessel between the vessel and each transducer, and
wherein the internal waterproof structure seals the vessel configured to prevent from being occurred leakage into the housing space.

11. The acoustic imaging apparatus according to claim 1, wherein each transducer has a longitudinal direction, the transducer including the corresponding reception surface at one end in the longitudinal direction and being provided with the corresponding signal line at the other end in the longitudinal direction.

12. The acoustic imaging apparatus according to claim 11, wherein each transducer includes a capacitive micromachined ultrasonic transducer on the corresponding reception surface.

13. An acoustic imaging apparatus comprising:
a container-shaped detector on which a plurality of ultrasonic transducers that receive acoustic waves generated by a subject are arranged,
wherein the acoustic imaging apparatus forms an image based on outputs from the ultrasonic transducers,
wherein an impedance matching material is disposed in the detector and a container-shaped cover member is disposed outside the detector, the detector and the cover member forming a sealed space, and
wherein signal lines of the ultrasonic transducers extend from the sealed space to the outside through a first waterproof member.
